(19) United States
(12) Patent Application Publication
(10) Pub. No.: US 2006/0041315 A1
(43) Pub. Date: Feb. 23, 2006

(54) Subtalar implant

(75) Inventors: Gary E. Katz, Gulfport, FL (US); David V. Mrak, North Street, MI (US); Steven E. Thueme, Columbus, MI (US)

Correspondence Address: BROOKS KUSHMAN P.C., 1000 TOWN CENTER, TWENTY-SECOND FLOOR, SOUTHFIELD, MI, 48075, US

(73) Assignee: BioPro, Inc., Port Huron, MI (US)

(21) Appl. No.: 11/245,753

(22) Filed: Oct. 7, 2005

Related U.S. Application Data
(63) Continuation-in-part of application No. 10/838,679, filed on May 4, 2004.

(51) Int. Cl. A61F 2/42

(52) U.S. Cl. 623/21.11

(57) ABSTRACT

An implant for insertion into a tarsal sinus includes a metal body having a plurality of threads disposed thereon. The body also includes a generally smooth, non-threaded portion that provides an articulating surface for the bones of the joint. The non-threaded portion tapers along a length, decreasing in size toward the tarsal sinus opening. This configuration mimics the shape of the tarsal canal when the foot is bearing weight, and therefore distributes the weight over a relatively large surface area.

### CROSS-REFERENCE TO RELATED APPLICATION

This application is a continuation-in-part of U.S. application Ser. No. 10/838,679 filed May 4, 2004.

### BACKGROUND OF THE INVENTION

1. Field of the Invention

The present invention relates to a subtalar implant, and in particular, an implant that can be used to correct a valgus deformity of the foot.

2. Background Art

For many years, surgeons have been attempting to correct valgus deformities of the foot—e.g., pes planus, or flatfoot—using a number of different techniques. One technique is to use a subtalar implant that is inserted into the tarsal sinus to reposition the calcaneus relative to the talus. One such device is described in U.S. Pat. No. 6,168,631 issued to Maxwell, et al. on Jan. 2, 2001. The implant described in Maxwell et al. is a metallic screw having external threads with slots formed in the threads. One limitation of the implant described in Maxwell, et al., is that there is no smooth surface on which the ankle bones can articulate. In fact, the talus and the calcaneus articulate on the same sharp-edged threads that are used to secure the implant within the joint. Such a configuration may lead to irritation of the articular bone and surrounding tissue.

In addition to not providing a smooth surface for bone articulation, the Maxwell implant has a generally uniform diameter, which may not accommodate the shape of the tarsal sinus. For example, when a patient is at rest—i.e., with no appreciable weight bearing on the affected foot—the tarsal sinus may be generally tapered: smaller inside the tarsal canal, and larger toward the canal opening on the lateral side of the ankle. Conversely, when there is weight bearing on the foot, the tarsal canal tends to form a reverse taper such that the lateral opening is reduced or effectively closed. Thus, it would be desirable to have a subtalar implant which matched the “reverse taper” of the tarsal canal so as to distribute the patient's weight over a greater surface area, thereby reducing stress on the bones.

Another subtalar implant is described in U.S. Pat. No. 5,360,450 issued to Giannini on Nov. 1, 1994. Giannini describes an implant configured for insertion into the tarsal sinus for correction of pes planus. The Giannini implant is a two-piece device consisting of a cylindrical body and a screw which is configured for insertion into the cylindrical body. The cylindrical body includes a longitudinal incision which allows the body to expand when the screw is inserted into an axial hole. The expansion of the body inside the tarsal canal tends to increase the thickness of the distal portion of the implant, while the thickness toward the proximal end of the implant—i.e., toward the lateral side of the ankle—remains essentially constant. The entire implant is made from a bioresorbable material, such that removal of the implant is not necessary; rather, it is designed to be resorbed into the patient's body.

Although the Giannini implant does not require the ankle bones to articulate on metal threads, it nonetheless has a number of limitations. For example, the outer surface of the cylindrical body includes a plurality of grooves which are intended to provide a location for the growth of fibrous tissue. Necessarily, a plurality of rings abut the grooves such that the bones do not have a smooth surface on which to articulate. In addition, the implant described in Giannini relies on a wedge-effect using a bioresorbable material to secure the implant. Thus, the Giannini implant does not have the benefit of threads to securely hold the implant in the joint space. Moreover, the wedge formed by the body ends near the opening of the tarsal canal; this does not match the typical shape of the canal during weight bearing. In fact, the Giannini implant includes an annular flange at its proximal end, which provides an increase in the implant diameter at a point where a smaller diameter is desirable. To form the wedge shape, the body is split; this leaves gaps on either side of the implant. These gaps are bounded by edges of the body which further impose a rough surface over which the bones articulate.

One implant which uses a combination of metallic and polymeric components is described in U.S. Pat. No. 6,136,032 issued to Viladot Perice et al. on Oct. 24, 2000. The implant described in Viladot Perice et al. is a three-piece implant that is configured for insertion into the tarsal sinus. The Viladot Perice et al. implant includes a metal cone which is drawn up toward an implant head, thereby expanding an outer polyethylene cylinder. The polyethylene cylinder includes a plurality. of fins which are shaped as barbs, tapering away from an outer surface of the cylinder and returning abruptly to the cylinder, thereby creating a sharp edge.

The implant described in Viladot Perice et al. relies on a wedge-effect and the polyethylene fins to secure the implant. Thus, Viladot Perice et al. implant does not have the advantage of threads to secure the implant within the joint space. Moreover, the Viladot Perice et al. implant includes a plurality of sharp-edged fins which may irritate the joint tissue as the bones articulate. In addition, this implant has a taper which increases in size from inside the tarsal canal to the outside, on lateral side of the ankle. This is the opposite of the typical shape of the canal during weight bearing.

Therefore, a need exists for an implant which can be used within the tarsal sinus that provides the advantage of threads to secure the implant in the joint space, and at the same time, provides a smooth surface on which the bones may articulate, thereby inhibiting friction and irritation within the joint. A need also exists for an implant which provides a taper to accommodate the shape of the tarsal canal when the foot is used for weight bearing.

### SUMMARY OF THE INVENTION

Accordingly, the present invention provides an implant that can be used in the tarsal sinus and includes metal threads which secure the implant in the joint, and also includes a polymeric portion having a smooth surface on which the bones can articulate, thereby inhibiting friction and irritation of the joint tissue.

The invention further provides a subtalar implant which includes a generally smooth, non-threaded portion having a taper which increases in size going into the tarsal canal, thereby generally matching the shape of the canal when the foot is bearing weight, and thus providing a relatively large surface area over which the weight is distributed.

The invention also provides an implant for insertion into a joint between articulating bones. The implant includes a generally cylindrical metal body having a proximal end and a distal end, and defining a longitudinal axis. The body includes at least one thread disposed on an external surface thereof. The at least one thread is configured to engage tissue in the joint. A generally smooth polymeric portion is.disposed adjacent the proximal end of the body. The polymeric portion includes an external surface configured to be disposed between articulating bones of the joint, thereby providing a bearing surface for the articulating bones.

The invention further provides an implant for insertion into a joint between articulating bones. The implant includes a generally cylindrical metal body having a proximal end and a distal end, and defining a longitudinal axis. The body includes at least one thread disposed on an external surface thereof. The at least one thread is configured to engage tissue in the joint, and to draw the implant into the joint when the body is rotated in one direction about the longitudinal axis. The body further includes a first axial hole disposed therethrough and generally parallel to the longitudinal axis. A polymeric portion is disposed adjacent the proximal end of the body. The polymeric portion includes an external surface configured to be disposed between articulating bones of the joint, thereby providing a bearing surface for the articulating bones. The polymeric portion further includes a second axial hole disposed therethrough and generally parallel to the longitudinal axis. An elongate member is disposed through the first and second axial holes. The elongate member has a proximal end and a distal end. The proximal end includes a first recess having at least one generally flat side to facilitate a rotation of the implant in one direction about the longitudinal axis for insertion into the joint.

The invention also provides a method of producing an implant for insertion into a joint between articulating bones. The implant includes a metal body and a polymeric portion. The metal body has at least one thread disposed on an external surface thereof, and the polymeric portion includes an external surface configured to be disposed between articulating bones in the joint, thereby providing a bearing surface for the articulating bones. The method includes disposing the polymeric portion adjacent a proximal end of the body such that a first axial hole in the body is generally aligned with a second axial hole in the polymeric portion. A pin is inserted through the first and second axial holes, and is secured to at least one of the polymeric portion and the body.

The invention further provides a subtalar implant for insertion into a tarsal sinus. The implant includes a threaded portion including at least one thread disposed on an external surface thereof for engaging tissue in the tarsal sinus. The implant also includes a generally smooth, non-threaded portion having a proximal end configured to be disposed adjacent an opening of the tarsal sinus and defining a first dimension, and a distal end disposed adjacent the threaded portion and defining a second dimension larger than the first dimension.

The invention also provides a subtalar implant for insertion into a tarsal sinus. The implant includes a one-piece body having a proximal end configured to be disposed adjacent an opening of the tarsal sinus, and a distal end configured to be disposed inside the tarsal sinus away from the opening. The body includes a threaded portion having at least one thread disposed thereon for engaging tissue in the tarsal sinus. The body also includes a generally smooth, non-threaded portion forming a taper along a predetermined length. The taper increases in size along the length from the proximal end of the body toward the threaded portion.

### BRIEF DESCRIPTION OF THE DRAWINGS

FIG. 1 is a side plan view of an implant in accordance with one embodiment of the present invention;

FIG. 2 is a sectional view of the implant shown in FIG. 1, taken through line 2-2;

FIG. 3 is a side plan view of the bones of the foot illustrating the implant of FIG. 1 disposed in the tarsal sinus;

FIG. 4 is a top plan view of the foot bones and implant shown in FIG. 3;

FIG. 5 is a side plan view of a body portion of the implant shown in FIG. 1;

FIG. 6 is a side sectional view of the body portion shown in FIG. 5, taken through line 6-6;

FIG. 7 is a side sectional view of a polymeric portion of the implant shown in FIG. 1;

FIG. 8 is a bottom plan view of the polymeric portion shown in FIG. 7;

FIG. 9 is a side sectional view of a pin shown in FIG. 2;

FIG. 10 is a top plan view of the pin shown in FIG. 9;

FIGS. 11A and 11B show a surgical tool that can be used to insert the implant shown in FIG. 1;

FIG. 12 shows an implant in accordance with a second embodiment of the present invention, and a guide tool that can be used to help locate the implant within the joint;

FIG. 13 shows an implant in accordance with a third embodiment of the present invention; and

FIGS. 14A and 14B show the implant shown in FIG. 13 implanted in a tarsal sinus having the foot at rest, and with weight bearing, respectively.

### DETAILED DESCRIPTION OF THE PREFERRED EMBODIMENT(S)

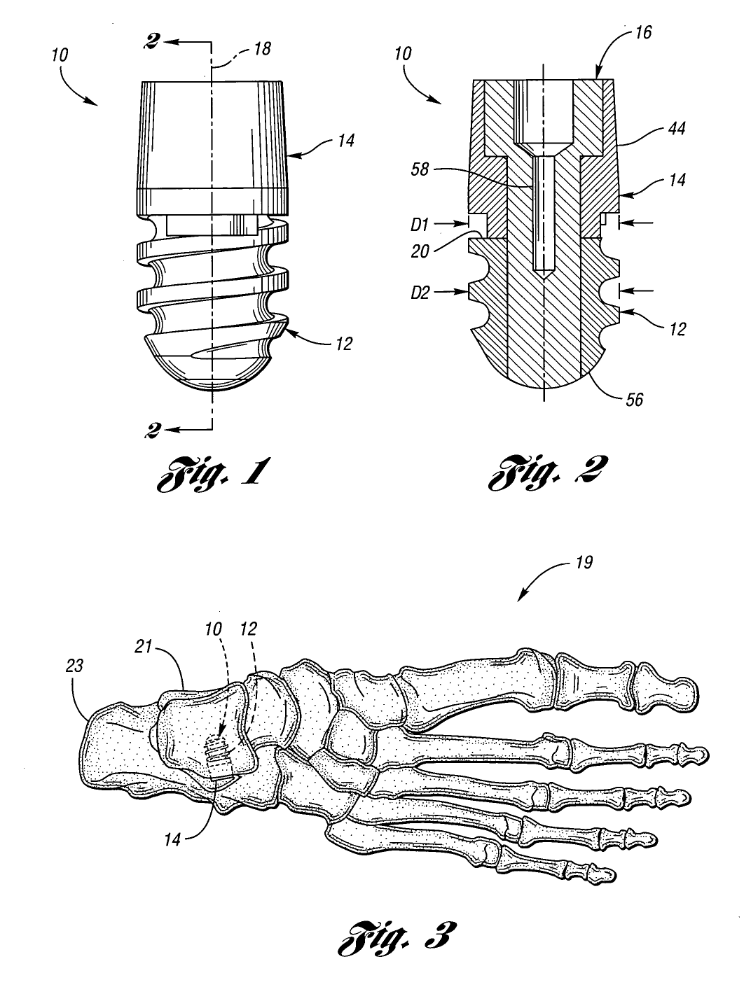

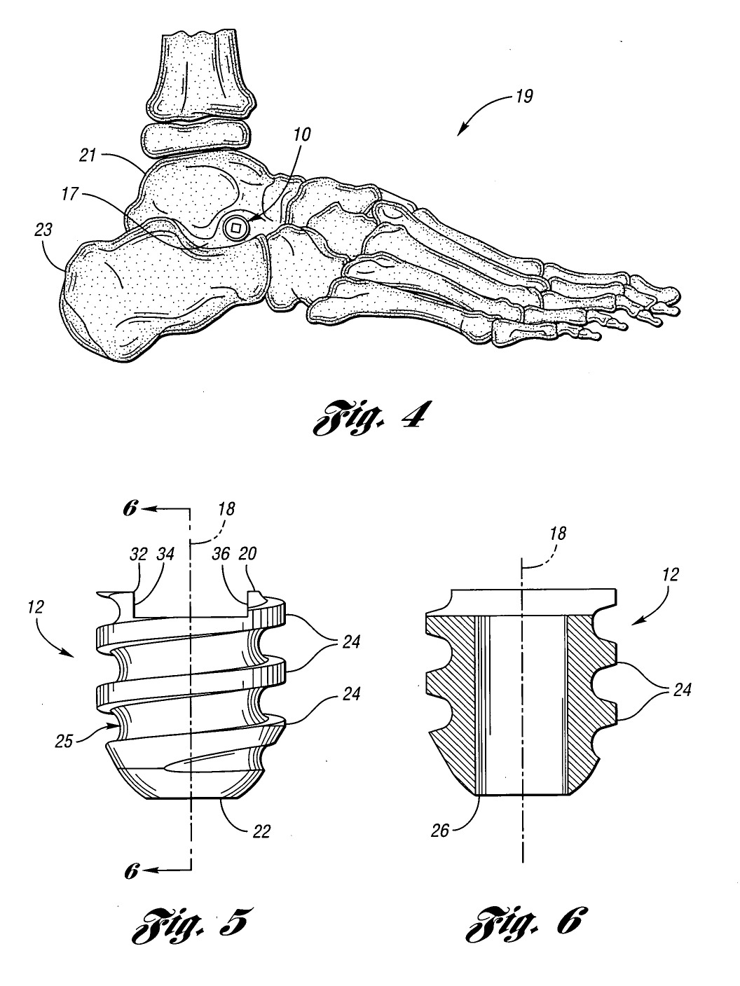

FIG. 1 shows an implant 10 in accordance with one embodiment of the present invention. The implant 10 includes a metal body 12 and a polymeric portion 14. FIG. 2 shows a section of the implant 10 taken through line 2-2 in FIG. 1. As shown in FIG. 2, the implant 10 also includes an elongate member, or pin 16. The implant 10 is sized to fit in a joint in the human body, and in particular, a tarsal sinus 17 of a foot 19, as illustrated in FIG. 3. Of course, an implant, such as the implant 10, may be provided in different sizes to fit within different joints in the body. As shown in FIGS. 3 and 4, the implant 10 is inserted between a talus 21 and a calcaneus 23 of the foot 19. As described more fully below, the body 12 secures the implant 10 in the tarsal sinus 17, while the polymeric portion 14 provides a smooth bearing surface on which the talus 21 and the calcaneus 23 can articulate.

FIG. 5 shows a front plan view of the body 12. The body 12 has a generally cylindrical shape, and defines a longitudinal axis 18. The body 12 includes a proximal end 20 and a distal end 22, and also includes a plurality of threads 24 disposed on an external surface 25. It is worth noting that the present invention contemplates the use of smaller implants, which may include a single thread, rather than the plurality of threads 24 shown in FIG. 5. The threads 24 are configured to engage tissue in the tarsal sinus 17—see FIGS. 3 and 4—thereby securely fixating the implant 10 within the joint. As the implant 10 is rotated about the longitudinal axis 18, the body 12 is also rotated. As the body 12 is rotated, the threads 24 engage tissue within the joint and draw the implant 10 into the joint space.

FIG. 6 shows a sectional view of the body 12 taken through line 6-6 in FIG. 5. In this view, it is shown that the body 12 includes a first axial hole 26 that is generally parallel to the longitudinal axis 18. The first axial hole 26 is a through-hole that is configured to receive the pin 16. As explained more fully below, the pin 16 keeps the body 12 and the polymeric portion 14 secured to each other. The body 12 can be made from any metal that is effective to secure the implant 10 within a joint and is generally indicated for implantation into the human body. For example, titanium and titanium alloys, cobalt-chromium alloys, and stainless steel alloys may be used. In particular, a titanium alloy conforming to the American Society For Testing And Materials (ASTM) Standard F136 may be used.

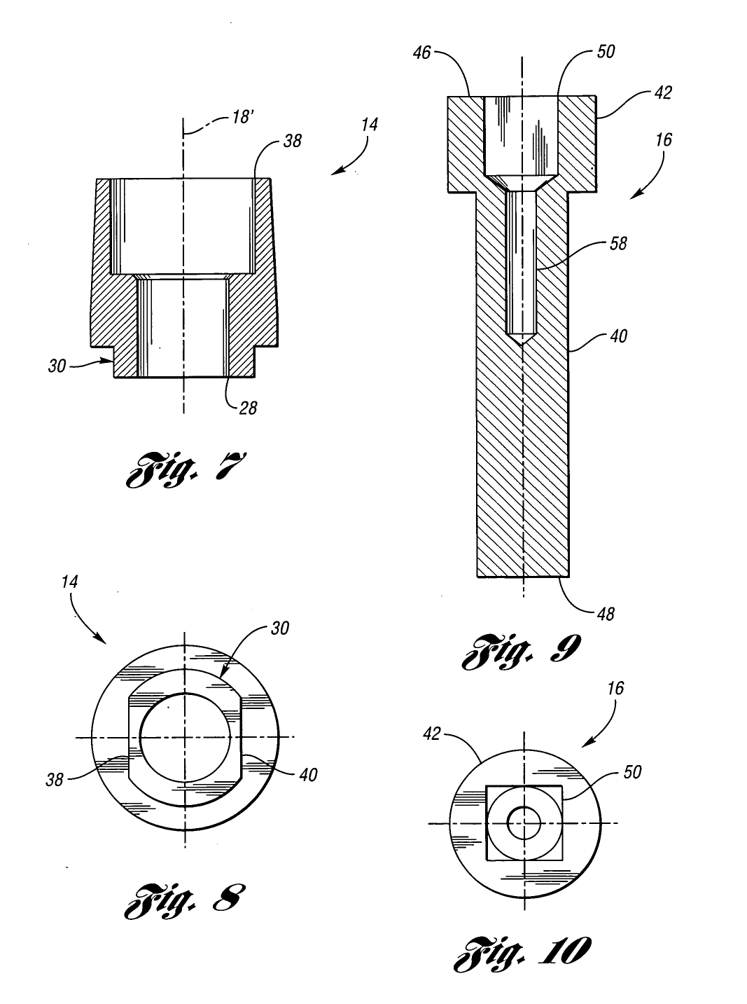

FIG. 7 shows a side plan view of the polymeric portion 14. In this view, the polymeric portion 14 is shown as a section, which illustrates a second axial hole 28 which is generally parallel to the longitudinal axis 18′ of the polymeric portion 14. The label 18′ is used to designate the longitudinal axis of the polymeric portion 14, since it is not associated with the body 12; however, when the implant 10 is assembled, such as shown in FIG. 1, the axes 18, 18′ will be generally coincident. This means that when the implant 10 is assembled, the first and second axial holes 26, 28 will be in general alignment, such that the pin 16 can be inserted through both the polymeric portion 14 and the body 12.

The polymeric portion 14 is configured to cooperate with a proximal end 20 of the body 12. In particular, the polymeric portion 14 includes a projection 30 which is configured to cooperate with a recess 32 in the body 12—see FIG. 5. As shown in FIG. 5, the recess 32 includes two flat sides 34, 36. The flat sides 34, 36 engage corresponding flat sides 38, 40 on the polymeric portion 14—see FIG. 8. The cooperation of the projection 30 and the recess 32 helps to keep the polymeric portion 14 rotationally stable after the implant 10 is inserted into the joint. In particular, the implant 10 is rotated about the longitudinal axis 18 as it is inserted into the joint. Once it is within the joint, the threads 24 engage the joint tissue which inhibits further rotation of the body 12 even when the bones in the joint articulate. The cooperation of the projection 30 on the polymeric portion 14, and the recess 32 in the body 12, inhibits rotation of the polymeric portion 14 about the longitudinal axis 18. This provides additional stability to the implant 10, and helps to reduce wear inside the polymeric portion 14. This may increase the longevity of the implant 10, as well as reduce irritation of the joint tissue.

As shown in FIG. 7, the polymeric portion 14 includes a recess 38 which is configured to receive a portion of the pin 16. In particular, turning to FIG. 9, the pin 16 includes a shank 40 and a head 42. The recess 38 in the polymeric portion 14 is configured to receive the head 42 of the pin 16. The recess 38 is made deep enough so that the head 42 does not extend substantially beyond the polymeric portion 14 after the pin 16 is inserted through the first and second axial holes 26, 28—see FIG. 2. As shown in FIG. 2, the polymeric portion 14 is disposed adjacent the proximal end 20 of the body 12. The polymeric portion 14 includes a smooth external surface 44 that is configured to be disposed between articulating bones in a joint, such as the talus 21 and the calcaneus 23—see FIGS. 3 and 4. This provides a smooth bearing surface for the articulating bones.

The polymeric portion 14 can be made from any polymer that provides a good wear surface and is generally indicated for implantation into the human body. For example, ultra-high-molecular-weight polyethylene, conforming to ASTM Standard F648 may be used, though other polymeric materials are contemplated within the scope of the invention. As shown in FIG. 2, the polymeric portion 14 has a maximum diameter (D1) that is approximately equal to, but no greater than, the maximum diameter (D2) of the body 12. Moreover, the polymeric portion 14 is tapered along a length leading away from the body 12. Having the maximum diameter of the polymeric portion 14 less than or equal to the maximum diameter of the body 12 facilitates insertion of the implant 10 into the joint. In particular, it allows the implant 10 to be inserted without the polymeric portion 14 unnecessarily impinging upon joint tissue as the implant 10 is being inserted.

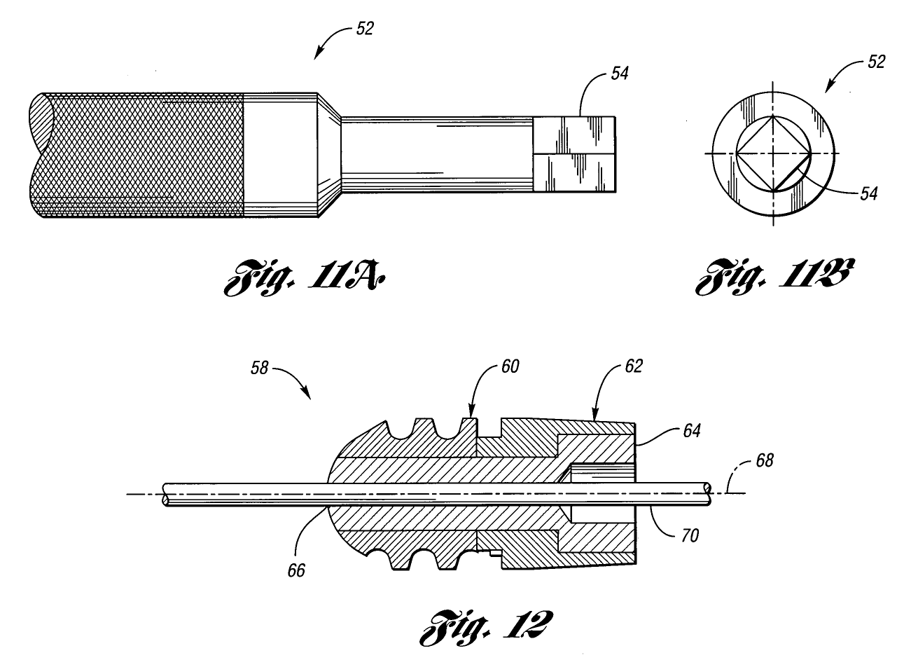

As shown in FIG. 9, the pin 16 includes a proximal end 46 and a distal end 48. The proximal end 46 of the pin 16 includes a recess 50 which has a generally square configuration—this is shown in FIG. 10, which is a top plan view of the pin 16. The square recess 50 is configured to receive a tool, such as the square-head driver 52 shown in FIGS. 11A and 11B. In fact, the recess 50 is an attachment feature which cooperates with the head 54 of the driver 52 to facilitate rotation of the implant 10 about the longitudinal axis 18, such that the implant 10 can be inserted into the joint. Although the embodiment of the pin 16 illustrated in the drawing figures includes a recess having four generally flat sides, other configurations are possible. For example, a generally circular recess having a single flat side would still facilitate rotation of an implant, such as the implant 10 about a longitudinal axis.

In order to produce an implant, such as the implant 10, the polymeric portion 14 is disposed adjacent the proximal end 20 of the body 12. The polymeric portion 14 and the body 12 can be attached to each other in any of a number of ways, and the use of the pin 16 provides one convenient and effective method. The pin 16 can be inserted through the first and second axial holes 26, 28, and specifically configured to be long enough to project beyond the proximal end 26 of the body 12. This facilitates welding the proximal end 48, or some portion of the pin 16 near the proximal end 48, to the proximal end 26 of the body 12. Welding the pin 16 to the body 12 provides the advantage of a strong attachment, and also seals the distal end 22 of the body 12 to prevent ingress of joint tissue and fluids.

Of course, welding the pin 16 to the body 12 requires that both components be made from materials that are compatible for welding. For example, if, as discussed above, the body 12 is made from a titanium alloy, it may be convenient to manufacture the pin 16 from the same alloy to ensure compatibility. After the pin 16 is welded to the body 12, the respective distal ends 48, 22 can be ground to a smooth radius, thereby creating an generally spherical end 56 as shown in FIG. 2. Although the embodiments illustrated in the drawing figures show a three-piece implant, the present invention contemplates the use of a two-piece implant having a metal body and polymeric portion attached to each other by any effective method, for example, a snap fit. The three-piece configuration illustrated in the drawing figures facilitates manufacturing and provides a fast and effective means of assembling an implant, such as the implant 10.

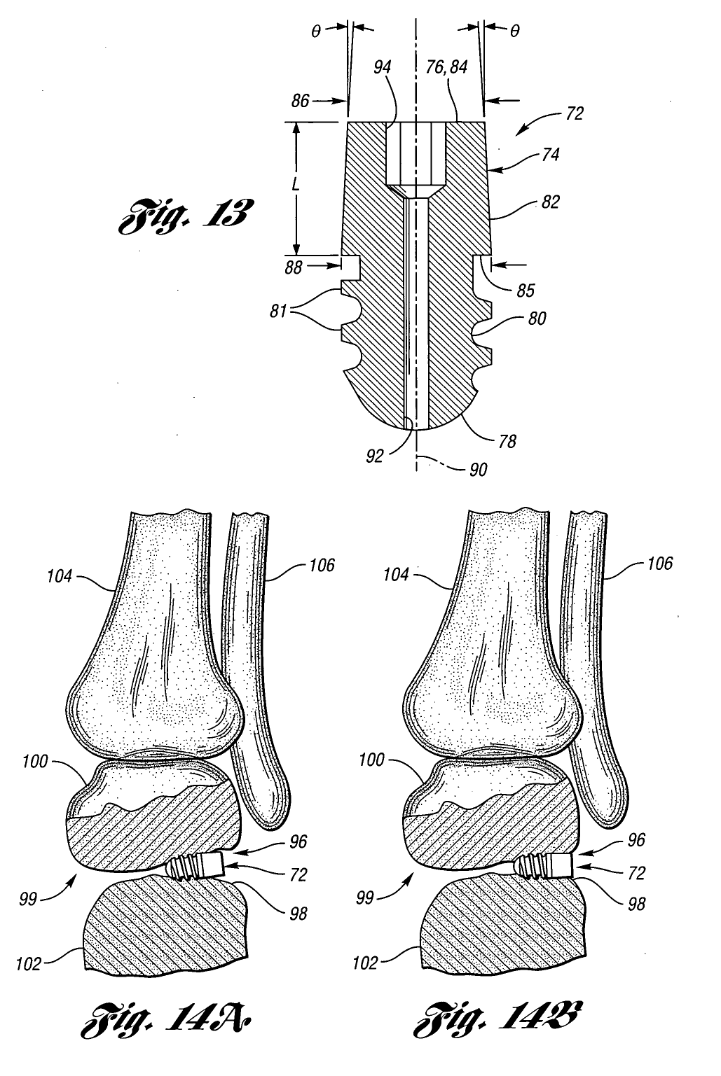

As shown in FIGS. 2 and 9, the pin 16 includes a blind hole 58 which may be included to facilitate manufacture of the recess 50. Alternatively, the hole can be disposed through the entire length of the pin, such as the pin 16. FIG. 12 shows an implant 58 in accordance with an alternative embodiment of the present invention. As with the implant 10, the implant 58 includes a body 60, a polymeric portion 62, and a pin 64. The pin 64 includes an aperture, or axial hole 66, which is disposed through the entire length of the pin 64. The axial hole 66 is generally parallel to a longitudinal axis 68 of the implant 58. The axial hole 66 is configured to receive a guide tool 70 which may be conveniently used by a surgeon to facilitate location and implantation of the implant 58 into the joint. The guide tool 70 may be a metal wire or other device which has the strength and flexibility to allow a surgeon to accurately guide the implant into the joint. Thus, the present invention provides an implant that is secured within the joint by metal threads, provides a smooth polymeric surface to inhibit friction and irritation of the joint, and is also easily located and inserted into the joint.

In addition to the embodiments described above, the present invention also contemplates an implant, such as subtalar implant 72, shown in FIG. 13. The implant 72 has a contour which is similar to the implants 10 and 58, shown in FIGS. 1 and 12, respectively. The implant 72 includes a one-piece body 74, though the body 74 could be made from more than one piece. The body 74 can be made from metal, for example, the titanium alloy described above. Alternatively, the body 74 can be made from other materials, such as polymers and ceramics. Moreover, even though the body 74 is of unitary construction, it may be made from more than one material, for example, two different materials could be combined in a molding process to form the one-piece body 74.

The body 74 has a proximal end 76 and a distal end 78. In addition, it includes a threaded portion 80 having a plurality of threads 81, and a generally smooth, non-threaded portion 82. The non-threaded portion 82 includes a proximal end 84, which coincides with the proximal end 76 of the body 74, and a distal end 85, which is adjacent to the threaded portion 80. In the embodiment shown in FIG. 13, the non-threaded portion 82 is frustoconical in shape. Thus, the proximal end 84 of the non-threaded portion 82 defines a first dimension, or first diameter 86. Similarly, the distal end 85 of the non-threaded portion 82 defines a second dimension, or second diameter 88, which is larger than the first diameter 86. The first and second diameters 86, 88 are separated by a length (L) of the non-threaded portion 82. In the embodiment shown in FIG. 13, the length (L) is the entire length of the non-threaded portion 82. This is not required, however, and the taper could, for example, stop before reaching the distal end 85 of the non-threaded portion 82.

As shown in FIG. 13, the second diameter 88 is the maximum diameter of the non-threaded portion 82. It is no greater than the maximum diameter of the threaded portion 80. As discussed above in relation the implant 10, this facilitates insertion of implant into the joint space. The first and second diameters 86, 88 and the length (L), define the taper of the non-threaded portion 82, and in particular, define the taper half-angle θ. Although the taper half-angle can be any angle effective for the particular patient, taper half-angles of 15°-45° have been found to be effective. Also shown in FIG. 13, the body 74 defines a longitudinal axis 90, and includes an aperture 92 disposed along the axis 90. The aperture 92 conveniently provides access for a tool, such as the guide tool 70, shown in FIG. 12. The proximal end 76 includes an attachment feature 94, which in the embodiment shown in FIG. 13, is a hexagonal opening which accommodates a hexagonal driver to facilitate insertion of the implant 72 into the joint.

By providing a taper which increases in size going into the tarsal canal, the implant 72 generally conforms to the shape of the canal when the affected foot is bearing weight. Conversely, the taper helps to ensure that contact with the implant 72 is minimized when there is little or no weight on the affected foot. FIG. 14A shows the implant 72 implanted in a tarsal sinus 96, having an opening 98 toward the lateral side of ankle 99. As shown in FIG. 14A, the implant 72 is disposed between a talus 100 and a calcaneus 102, with a tibia 104 and fibula 106 shown for reference.

The ankle 99 shown in FIG. 14A is “at rest”—i.e., it is not exposed to any significant weight bearing. In the at-rest position, the tarsal sinus 96 has a generally decreasing taper from the opening 98, inward toward the center of the joint. Thus, although the threads 81 keep the implant 72 secured in the joint, the non-threaded portion 82 has limited contact with the talus 100 and the calcaneus 102. This may help to reduce joint irritation as compared to implants which are of constant diameter or have an increasing taper outward to the opening of a tarsal sinus.

In contrast to FIG. 14A, FIG. 14B shows the ankle 99 in a weight bearing posture. In this position, the tarsal sinus 96 has reversed its taper from the non-weight bearing posture. In this position, the taper of the non-threaded portion 82 of the implant 72 generally conforms to the taper of the tarsal sinus 96. This helps to ensure a relatively large area of contact between the non-threaded portion 82 and the talus 100 and calcaneus 102. Such a configuration distributes the weight over a larger surface area, thereby reducing stress on the talus 100 and the calcaneus 102. This provides an advantage over subtalar implants having constant diameters or tapers which increase toward the joint opening.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A subtalar implant for insertion into a tarsal sinus, the implant comprising:
a threaded portion including at least one thread disposed on an external surface thereof for engaging tissue in the tarsal sinus; and
a generally smooth, non-threaded portion having a proximal end configured to be disposed adjacent an opening of the tarsal sinus and defining a first dimension, and a distal end disposed adjacent the threaded portion and defining a second dimension larger than the first dimension.

2. The implant of claim 1, wherein the non-threaded portion is generally frustoconical, and wherein the first and second dimensions are respectively first and second diameters.

3. The implant of claim 2, wherein the non-threaded portion has a maximum diameter that is no greater than a maximum diameter of the threaded portion, thereby facilitating insertion of the implant into the tarsal sinus.

4. The implant of claim 2, wherein the first and second diameters are separated by a first length, thereby forming a taper, the taper defining a taper half-angle between 15 degrees and 45 degrees.

5. The implant of claim 1, wherein the proximal end of the non-threaded portion includes an attachment feature configured to cooperate with a tool to facilitate rotation of the implant in one direction about the longitudinal axis for insertion into the tarsal sinus.

6. The implant of claim 1, wherein the threaded portion and the non-threaded portion form a unitary structure.

7. A subtalar implant for insertion into a tarsal sinus, the implant comprising:
a one-piece body having a proximal end configured to be disposed adjacent an opening of the tarsal sinus and a distal end configured to be disposed inside the tarsal sinus away from the opening, the body including:
a threaded portion having at least one thread disposed thereon for engaging tissue in the tarsal sinus, and
a generally smooth, non-threaded portion forming a taper along a predetermined length, the taper increasing in size along the length from the proximal end of the body toward the threaded portion.

8. The implant of claim 7, wherein the body defines a longitudinal axis and includes an aperture disposed therethrough, the aperture being generally parallel to the longitudinal axis and configured to receive a guide tool for facilitating insertion of the implant into the joint.

9. The implant of claim 7, wherein the non-threaded portion is generally frustoconical.

10. The implant of claim 9, wherein the non-threaded portion has a maximum diameter that is no greater than a maximum diameter of the threaded portion, thereby facilitating insertion of the implant into the tarsal sinus.

11. The implant of claim 7, wherein the taper defines a taper half- angle between 15 degrees and 45 degrees.

12. The implant of claim 7, wherein the proximal end of the body includes an attachment feature configured to cooperate with a tool to facilitate rotation of the implant in one direction about the longitudinal axis for insertion into the tarsal sinus.

13. The implant of claim 7, wherein the body includes at least one of a metal material and a ceramic material.
